(19) United States
(12) Patent Application Publication
(10) Pub. No.: US 2021/0085174 A1
(43) Pub. Date: Mar. 25, 2021

(54) EYEBALL STRUCTURE ESTIMATION APPARATUS

(71) Applicant: AISIN SEIKI KABUSHIKI KAISHA, Kariya-shi (JP)

(72) Inventors: Shin-ichi KOJIMA, Nagakute-shi (JP); Yoshiyuki YAMADA, Kariya-shi (JP)

(73) Assignee: AISIN SEIKI KABUSHIKI KAISHA, Kariya-shi (JP)

(21) Appl. No.: 16/817,697

(22) Filed: Mar. 13, 2020

(30) Foreign Application Priority Data
Sep. 25, 2019 (JP) 2019-174079

(51) Int. Cl. A61B 3/107; G06T 7/00; G06T 7/70; A61B 3/00; A61B 3/14

(52) U.S. Cl. CPC A61B 3/107; G06T 7/0012; G06T 2207/30041; A61B 3/0025; A61B 3/14; G06T 7/70

(57) ABSTRACT

An eyeball structure estimation apparatus includes: an imaging unit imaging a face of a person to be observed; a light irradiation unit irradiating an eye of the person with light; a corneal reflex estimation unit that estimates an image coordinate of a corneal reflex in a face image representing the face using structure parameters of an eyeball on the basis of a pupil center position; a pupil estimation unit estimating an image coordinate of a pupil center using the structure parameters on the basis of a position of the corneal reflex; an advance prediction unit predicting a state vector including the structure parameters; and a state estimation unit estimating the state vector on the basis of an observation vector including the image coordinates of the corneal reflex and the pupil center, the state vector, and an observation equation representing the observation vector by using the state vector.

### CROSS REFERENCE TO RELATED APPLICATIONS

This application is based on and claims priority under 35 U.S.C. § 119 to Japanese Patent Application 2019-174079, filed on Sep. 25, 2019, the entire contents of which are incorporated herein by reference.

### TECHNICAL FIELD

This disclosure relates to an eyeball structure estimation apparatus, and particularly to an eyeball structure estimation apparatus that estimates a parameter of an eyeball by using a captured image of a face.

### BACKGROUND DISCUSSION

In the related art, there is a visual line measurement method of gazing at a single point in personal calibration (JP Takehiko OHNO, “One-Point Personal Calibration and Its Applications” Information Processing Society of Japan, 2006-HI-117(10), January 2006).

There is a visual line detection computer program in which a parameter is changed starting from an initial parameter of an eyeball model, adaptation of a corneal reflex and an iris contour is evaluated, and changing of parameters is repeated until the adaptation is equal to or less than a threshold value (JP 2018-120299A).

There is a sleepiness level estimation apparatus that obtains a structure parameter through nonlinear optimization by using iris region images at a plurality of time points starting from an initial parameter of an eyeball model (JP 2013-202273A).

There is a correction value calculation apparatus using the fact that a target of which a coordinate is known has been viewed (Japanese Patent No. 05560858). In the correction value calculation apparatus, a translation and a scale of a visual line vector are corrected.

There is a visual line detection apparatus in which a plurality of eyeball models of which structure parameters are different from each other are prepared in advance, and a model suitable for observation of the pupil is selected (Japanese Patent No. 04829141).

In Takehiko OHNO, “One-Point Personal Calibration and Its Applications” Information Processing Society of Japan, 2006-HI-117(10), January 2006, two LEDs are required to be provided separately from each other, and thus an apparatus becomes large-sized. Two corneal reflexes are required to be observed.

JP 2018-120299A employs the method in which a parameter is changed, adaptation is evaluated, and changing of parameters is repeated until the adaptation is equal to or less than a threshold value, and thus time is taken for retrieval.

In JP 2013-202273A, corneal refraction is taken into consideration when a projection position of the iris center is computed, and thus an error is considerable. The method is employed in which a parameter is changed, adaptation is evaluated, and changing of parameters is repeated until the adaptation is equal to or less than a threshold value, and thus time is taken for retrieval.

In Japanese Patent No. 05560858, it is difficult to determine that a target of which a coordinate is known has been relatively viewed.

Japanese Patent No. 04829141 employs the method in which a structure parameter is selected through comparison among a plurality of models prepared in advance, and thus time is taken for retrieval.

Thus, a need exists for an eyeball structure estimation apparatus which is not susceptible to the drawback mentioned above.

### SUMMARY

An eyeball structure estimation apparatus according to an aspect of this disclosure includes an imaging unit that images a face of a person to be observed; a light irradiation unit that irradiates an eye of the person to be observed with light; a corneal reflex estimation unit that estimates an image coordinate of a corneal reflex in a face image representing the face imaged by the imaging unit by using structure parameters of an eyeball on the basis of a pupil center position of the eye in the face image; a pupil estimation unit that estimates an image coordinate of a pupil center in the face image by using the structure parameters of the eyeball on the basis of a position of the corneal reflex of the eye in the face image; an advance prediction unit that predicts a state vector including the structure parameters of the eyeball; and a state estimation unit that estimates the state vector on the basis of an observation vector including the image coordinate of the corneal reflex in the face image, estimated by the corneal reflex estimation unit, and the image coordinate of the pupil center in the face image, estimated by the pupil estimation unit, the state vector predicted by the advance prediction unit, and an observation equation representing the observation vector by using the state vector.

### BRIEF DESCRIPTION OF THE DRAWINGS

The foregoing and additional features and characteristics of this disclosure will become more apparent from the following detailed description considered with the reference to the accompanying drawings, wherein:

FIG. 1 is a diagram illustrating a three-dimensional eyeball model and structure parameters;

FIG. 2 is a block diagram illustrating a configuration of a visual line measurement apparatus according to an embodiment disclosed here;

FIG. 3 is a diagram illustrating disposition of an irradiation unit and an image capturing unit;

FIG. 4 is a diagram illustrating a positional relationship among the irradiation unit, the image capturing unit, and the eye;

FIG. 5 is a block diagram illustrating a configuration of a corneal reflex estimation unit of the visual line measurement apparatus according to the embodiment disclosed here;

FIG. 6 is a diagram for describing various coordinate systems;

FIG. 7 is a diagram illustrating a positional relationship between the three-dimensional eyeball model and the image capturing unit;

FIG. 8 is a block diagram illustrating a configuration of a pupil estimation unit of the visual line measurement apparatus according to the embodiment disclosed here;

FIG. 9 is a block diagram illustrating a configuration of a structure parameter estimation unit of the visual line measurement apparatus according to the embodiment disclosed here;

FIG. 10 is a flowchart illustrating contents of a visual line measurement process routine in the visual line measurement apparatus according to the embodiment disclosed here;

FIG. 11 is a flowchart illustrating contents of a corneal reflex estimation process routine in the visual line measurement apparatus according to the embodiment disclosed here; and

FIG. 12 is a flowchart illustrating contents of a pupil estimation process routine in the visual line measurement apparatus according to the embodiment disclosed here.

### DETAILED DESCRIPTION

Hereinafter, with reference to the drawings, an embodiment disclosed here will be described in detail. In the present embodiment, as an example, a description will be made of a case where this disclosure is applied to a visual line measurement apparatus that estimates a visual line vector by using a captured face image.

### Outline of Embodiment

In the related art, a visual line vector is calibrated by showing a person to be observed a three-dimensional target of which a coordinate is known, so that an error due to an individual difference is absorbed, and thus a structure parameter of a three-dimensional eyeball model is not estimated. However, time is taken for calibration procedures.

Therefore, in the embodiment disclosed here, a structure parameter of a three-dimensional eyeball model is estimated in time-series visual line measurement processes without performing explicit calibration procedures.

Here, there is a technique of estimating an eyeball center coordinate e (JP 2019-000136A). This technique uses the fact that an eyeball center coordinate eₘ in a face model coordinate system is a fixed point, and thus does not change over time. A formula can be established in the form of eₘ=f (observation variable), and thus the formula is solved according to a least square method in a time direction.

An estimation target in the present embodiment is a structure parameter of an eyeball, and does not change over time regardless of a coordinate system. However, deformation to a form of a state variable=f(observation variable) is not possible, and thus the least square method cannot be applied.

Instead, it has been found that a formula can be established in a form of an observation variable=f(state variable). Therefore, f( ) indicates that partial differentiation can be performed with a state variable, and is solved as a fixed parameter estimation problem using a Kalman filter, and thus a structure parameter is estimated.

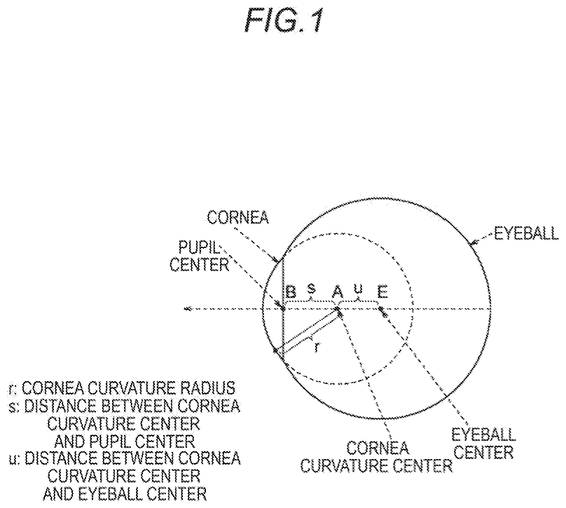

As illustrated in FIG. 1, a three-dimensional eyeball model is formed of two balls, and structure parameters estimated in the present embodiment are a cornea curvature radius r, a distance s between the cornea curvature center and the pupil center, and a distance u between the cornea curvature center and the eyeball center.

### Configuration of Visual Line Measurement Apparatus

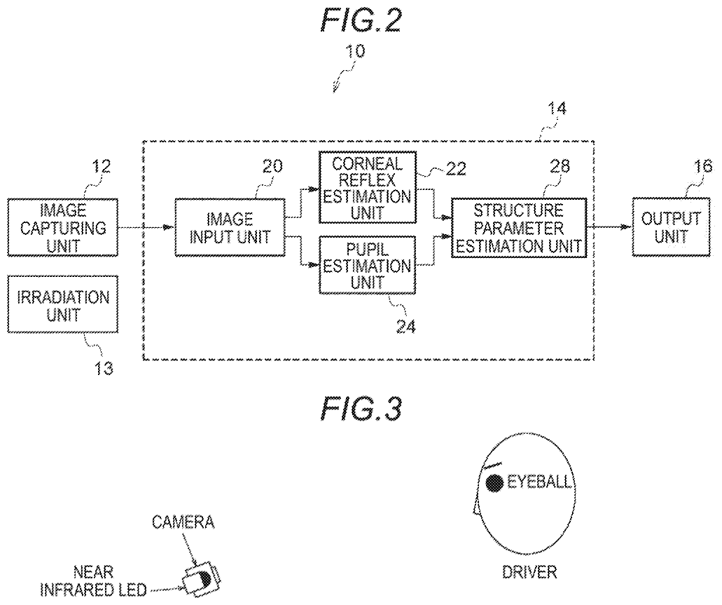

As illustrated in FIG. 2, a visual line measurement apparatus 10 according to the embodiment disclosed here includes an image capturing unit 12 formed of a CCD camera capturing an image including a face of a subject, an irradiation unit 13 that irradiates the eye of the subject with light, a computer 14 that performs image processing, and an output unit 16 formed of a CRT or the like.

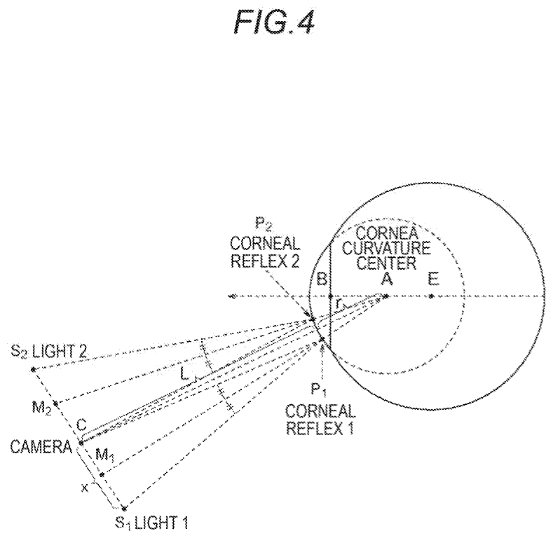

The image capturing unit 12 is a single camera, and the irradiation unit 13 is, for example, a single near infrared LED. In the present embodiment, an imaging direction of the image capturing unit 12 and an irradiation direction of the irradiation unit 13 are not present on the same axis, but the image capturing unit 12 and the irradiation unit 13 are disposed such that the imaging direction and the irradiation direction are regarded to be present on the same axis (FIG. 3). Specifically, the image capturing unit 12 and the irradiation unit 13 are disposed to satisfy a constraint condition shown in the following Equation (1) (FIG. 4). In a case where Equation (1) is satisfied, two near infrared LEDs may be disposed on both of the right and left sides of the camera.

$$x < \frac{L}{f} \frac{r + L}{r} \qquad ( 1 )$$

Here, L is a distance between an intersection and the image capturing unit 12, the intersection between a straight line from the image capturing unit 12 toward the cornea curvature center and the cornea, r is a cornea curvature radius, and f is a focal length of a pixel unit of the image capturing unit 12.

The computer 14 is configured to include a CPU, a ROM storing a program for a visual line measurement process routine which will be described later, a RAM storing data or the like, and a bus connecting the above-described elements to each other. In a case where the computer 14 is described as a functional block divided for each function realizing means that is defined on the basis of hardware and software, as illustrated in FIG. 2, the computer 14 includes an image input unit 20 that receives a face image which is a gradation image output from the image capturing unit 12, a corneal reflex estimation unit 22 that estimates a time series of image coordinates of a corneal reflex on the basis of a time series of face images output from the image input unit 20, a pupil estimation unit 24 that estimates a time series of image coordinates of the pupil center on the basis of the time series of face images output from the image input unit 20, and a structure parameter estimation unit 28 that estimates a structure parameter of an eyeball on the basis of the time series of image coordinates of the corneal reflex and the time series of image coordinates of the pupil center.

The image input unit 20 is formed of, for example, an ND converter and an image memory storing image data corresponding to a single screen.

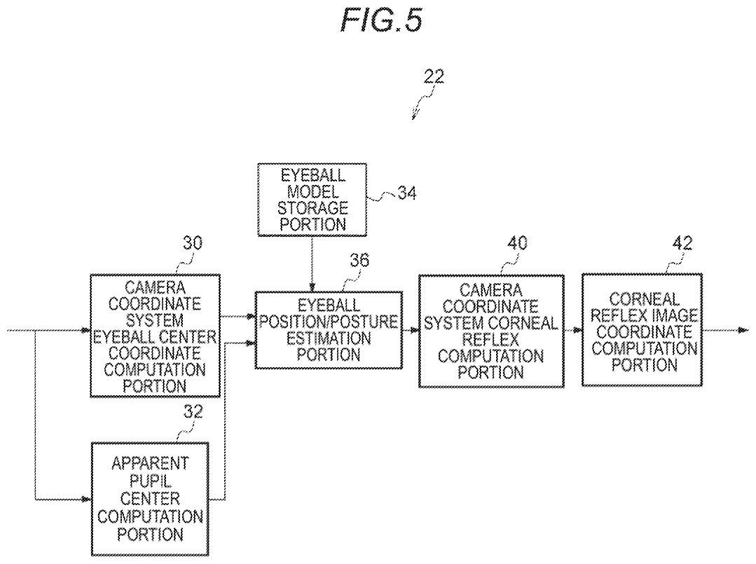

As illustrated in FIG. 5, the corneal reflex estimation unit 22 includes a camera coordinate system eyeball center coordinate computation portion 30, an apparent pupil center computation portion 32, an eyeball model storage portion 34, an eyeball position/posture estimation portion 36, a camera coordinate system corneal reflex computation portion 40, and a corneal reflex image coordinate computation portion 42.

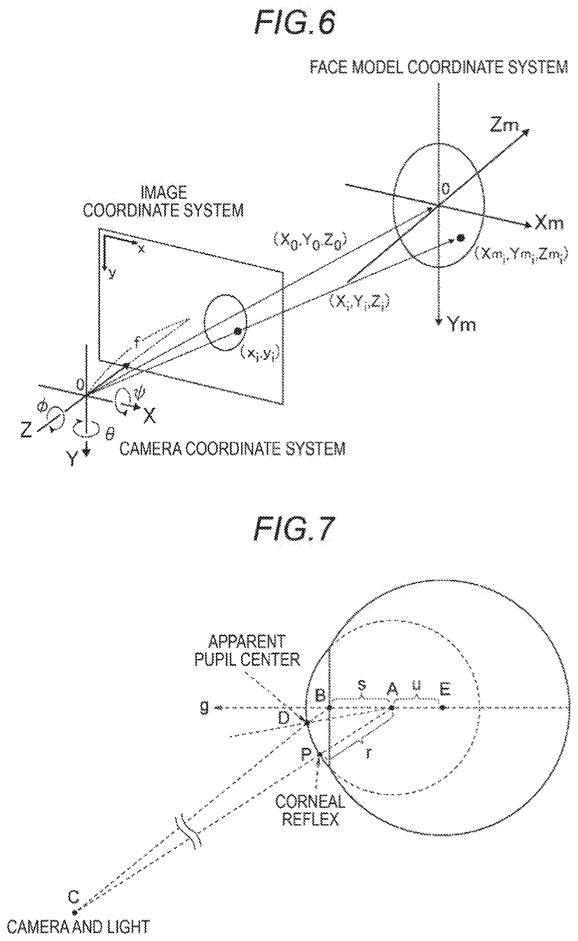

The camera coordinate system eyeball center coordinate computation portion 30 estimates a three-dimensional coordinate of the eyeball center in a camera coordinate system illustrated in FIG. 6 on the basis of a face image as follows.

First, a three-dimensional coordinate \vec{CE}=eₘ of the eyeball center (point E) in a face model coordinate system illustrated in FIG. 6 is obtained in advance (refer to FIG. 7).

For example, a visual line is computed by using the cornea curvature center and the pupil center that are computed on the basis of a corneal reflex of a face image, and a three-dimensional coordinate of the eyeball center in the eyeball model coordinate system is estimated by using the computed visual line.

A position and a posture (rotation and translation vectors) of the face model coordinate system in the camera coordinate system are obtained.

For example, a face model is fitted to the current face image, and thus the current rotation matrix R and the current translation vector t of the face model coordinate system with respect to the camera coordinate system are obtained.

A three-dimensional coordinate of the eyeball center in the face model coordinate system is converted into a three-dimensional coordinate of the eyeball center in the camera coordinate system by using the obtained rotation and translation vectors.

Specifically, the current three-dimensional coordinate e of the eyeball center in the camera coordinate system is computed according to the following equation.

e=Reₘ+t (2)

As will be described below, the apparent pupil center computation portion 32 estimates a three-dimensional coordinate of the apparent pupil center in the camera coordinate system on the basis of a pupil center position of the eye in the face image.

First, the pupil center is detected from the face image, and a pupil center coordinate in an image coordinate system illustrated in FIG. 6 is obtained.

Specifically, the pupil center is detected by using a known technique of the related art, and a pupil center coordinate D=(Dₓ,D_{y}) in the image coordinate system is obtained.

A three-dimensional coordinate in the camera coordinate system is estimated on the basis of the image coordinate of the pupil center.

Specifically, a Z coordinate of the pupil center in the camera coordinate system is obtained by using any range-finding means, and is indicated by d_{z}. A coordinate of the image center is indicated by (x_{c},y_{c}). A three-dimensional coordinate d (dₓ,d_{y},d_{z}) of the pupil center in the camera coordinate system is as follows in a case where a focal length represented in the pixel unit is indicated by f.

$$d = ( \frac{( {D}_{x} - {x}_{c} ) {d}_{z}}{f} , \frac{( {D}_{y} - {y}_{c} ) {d}_{z}}{f} , {d}_{z} ) \qquad ( 3 )$$

The eyeball model storage portion 34 stores an eyeball model formed of two balls and parameters thereof. Specifically, the eyeball model storage portion 34 stores an initial value or an estimated value of the cornea curvature radius r, an initial value or an estimated value of the distance u between the eyeball center E and the cornea curvature center A, an initial value or an estimated value of the distance s between the cornea curvature center A and the real pupil center B, and a ratio (n₁/n₂) between a refractive index n₁ of the atmosphere and a refractive index n₂ of the cornea. The distance s between the cornea curvature center A and the real pupil center B and the ratio (n₁/n₂) between the refractive index n₁ of the atmosphere and the refractive index n₂ of the cornea are parameters used when a visual line vector in the camera coordinate system is computed by the structure parameter estimation unit 28.

As will be described below, the eyeball position/posture estimation portion 36 computes a three-dimensional optical axis vector directed from a three-dimensional position of the eyeball center toward a three-dimensional position of the pupil center in the camera coordinate system on the basis of a three-dimensional coordinate of the eyeball center in the camera coordinate system and a three-dimensional coordinate of the apparent pupil center in the camera coordinate system.

First, since the image capturing unit 12 and the irradiation unit 13 are disposed such that an imaging direction of the image capturing unit 12 and an irradiation direction of the irradiation unit 13 are regarded to be present on the same axis, an angle correction amount is estimated by using a corner CED illustrated in FIG. 7.

Specifically, in a case where a vector from the eyeball center E to the apparent pupil center is indicated by \vec{ED}=g_{eb}, the vector is represented by the following equation.

$${g}_{eb} = d - e = ( \begin{matrix}\frac{( {D}_{z} - {y}_{c} ) {d}_{z}}{f} - {e}_{x} \\ \frac{( {D}_{y} - {y}_{c} ) {d}_{z}}{f} - {e}_{y} \\ {d}_{z} - {e}_{z}\end{matrix} ) \qquad ( 4 )$$

In a case where the corner CED formed by a line segment connecting a three-dimensional coordinate of the apparent pupil center in the camera coordinate system to a three-dimensional coordinate of the eyeball center in the camera coordinate system and a line segment connecting the three-dimensional coordinate of the eyeball center in the camera coordinate system to a three-dimensional coordinate of the imaging unit is indicated by ∠CED=ω, the angle ω of the corner CED is computed according to the following equation.

$$ω = arccos ( \frac{{g}_{eb}}{‖ {g}_{eb} ‖ ‖ - e ‖} ) = arccos ( \text{?} ) \\ \text{?} \text{indicates text missing or illegible when filed} \qquad ( 5 )$$

A relationship between an angle of the corner CED and an angle difference ρ between a visual line vector g_{cr} obtained according to a corneal reflex method and the visual line vector g_{eb} obtained according to an eyeball model fitting method is obtained in advance, and the angle difference ρ corresponding to an angle of the corner CED is obtained as a correction amount ρ by using the relationship.

A three-dimensional optical axis vector g directed from the three-dimensional coordinate of the eyeball center toward the three-dimensional coordinate of the pupil center in the camera coordinate system is computed on the basis of the three-dimensional coordinate e of the eyeball center in the camera coordinate system, the vector g_{eb} from the eyeball center E to the apparent pupil center, and the angle correction amount ρ according to the following equation.

$$g = {g}_{eb} cos ρ + ( \frac{- {g}_{eb} \times e}{‖ {g}_{eb} \times e ‖} ) \times {g}_{eb} sin ρ + ( \frac{- {g}_{eb} \times e}{‖ {g}_{eb} \times e ‖} ) ( \frac{- {g}_{eb} \times e}{‖ {g}_{eb} \times e ‖} \cdot {g}_{eb} ) ( 1 - cos ρ ) \qquad ( 6 )$$

As mentioned above, the optical axis vector is defined on the basis of the correction amount and the three-dimensional coordinate of the eyeball center, and a position and a posture of the eyeball model can be estimated through combination with the three-dimensional coordinate of the eyeball center.

As will be described below, the camera coordinate system corneal reflex computation portion 40 obtains a three-dimensional coordinate of a corneal reflex in the camera coordinate system on the basis of a three-dimensional coordinate of the eyeball center in the camera coordinate system, an optical axis vector, and a predefined three-dimensional eyeball model.

First, a three-dimensional coordinate a of the cornea curvature center A in the camera coordinate system is estimated according to the following equation by using the three-dimensional coordinate e of the eyeball center in the camera coordinate system, the optical axis vector g, and the distance u between the eyeball center E and the cornea curvature center A stored in the eyeball model storage portion 34.

$$α = e + u \frac{g}{‖ g ‖} \qquad ( 7 )$$

A point of which a distance is r from the cornea curvature center A on the vector CA is obtained.

Specifically, in a case where a three-dimensional vector of a corneal reflex P is indicated by p=(pₓ, p_{y}, p_{z}), the point P is a point on the C side by the length r from the point A on the straight line CA, and thus p is obtained according to the following Equation (8).

$$p = \frac{a}{‖ a ‖} ( ‖ a ‖ - r ) \qquad ( 8 )$$

As will be described below, the corneal reflex image coordinate computation portion 42 estimates an image coordinate of a corneal reflex in a face image on the basis of a three-dimensional coordinate of the corneal reflex in the camera coordinate system.

First, the three-dimensional coordinate of the corneal reflex in the camera coordinate system is two-dimensionally projected by using camera parameters.

Specifically, in a case where a focal length represented in the pixel unit is indicated by f, and a coordinate of the image center is indicated by (x_{c},y_{c}), an image coordinate (Pₓ,P_{y}) of the corneal reflex is as follows.

$$\begin{matrix}{P}_{x} = f \frac{{p}_{x}}{{p}_{z}} + {x}_{c} & ( 9 ) \\ {P}_{y} = f \frac{{p}_{y}}{{p}_{z}} + {y}_{c} & ( 10 )\end{matrix}$$

An image coordinate of a corneal reflex can be estimated by using observed values of the pupil center through the process in each portion.

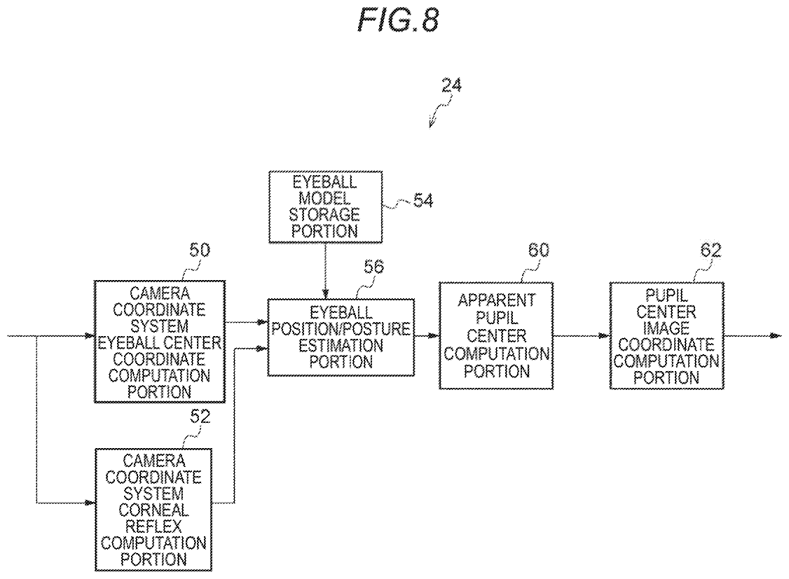

As illustrated in FIG. 8, the pupil estimation unit 24 includes a camera coordinate system eyeball center coordinate computation portion 50, a camera coordinate system corneal reflex computation portion 52, an eyeball model storage portion 54, an eyeball position/posture estimation portion 56, an apparent pupil center computation portion 60, and a pupil center image coordinate computation portion 62.

In the same manner as the camera coordinate system eyeball center coordinate computation portion 30, the camera coordinate system eyeball center coordinate computation portion 50 estimates a three-dimensional coordinate of the eyeball center in the camera coordinate system by using a face image.

The camera coordinate system corneal reflex computation portion 52 estimates a three-dimensional coordinate of a corneal reflex in the camera coordinate system on the basis of a position of the corneal reflex of the eye in a face image.

First, the corneal reflex is detected from the face image by using a known technique of the related art, and a coordinate P=(Pₓ,P_{y}) of the corneal reflex in the image coordinate system is obtained.

A three-dimensional coordinate of the corneal reflex in the camera coordinate system is estimated by using the image coordinate of the corneal reflex.

Specifically, a Z coordinate of the corneal reflex coordinate in the camera coordinate system is obtained by using any range-finding means, and is indicated by p_{z}. A coordinate of the image center is indicated by (x_{c},y_{c}). A three-dimensional coordinate p=(pₓ,p_{y},p_{z}) of the corneal reflex in the camera coordinate system is as follows in a case where a focal length represented in the pixel unit is indicated by f.

$$p = ( \frac{( {P}_{x} - {x}_{c} ) {p}_{z}}{f} , \frac{( {P}_{y} - {y}_{c} ) {p}_{z}}{f} , {p}_{z} ) \qquad ( 11 )$$

In the same manner as the eyeball model storage portion 34, the eyeball model storage portion 54 stores the cornea curvature radius r, the distance u between the eyeball center E and the cornea curvature center A, the distance s between the cornea curvature center A and the real pupil center B, and the ratio (n₁/n₂) between the refractive index n₁ of the atmosphere and the refractive index n₂ of the cornea. The distances between the cornea curvature center A and the real pupil center B and the ratio (n₁/n₂) between the refractive index n₁ of the atmosphere and the refractive index n₂ of the cornea are parameters used when a visual line vector in the camera coordinate system is computed by the structure parameter estimation unit 28.

As will be described below, the eyeball position/posture estimation portion 56 computes a three-dimensional optical axis vector directed from a three-dimensional position of the eyeball center toward a three-dimensional position of the cornea curvature center in the camera coordinate system on the basis of a three-dimensional coordinate of the eyeball center in the camera coordinate system and a three-dimensional coordinate of the corneal reflex in the camera coordinate system.

First, since the image capturing unit 12 and the irradiation unit 13 are disposed such that an imaging direction of the image capturing unit 12 and an irradiation direction of the irradiation unit 13 are regarded to be present on the same axis, a three-dimensional coordinate of the cornea curvature center A in the camera coordinate system is estimated by using the three-dimensional coordinate p of the corneal reflex in the camera coordinate system and the cornea curvature radius r.

Specifically, in a case where a three-dimensional vector of the cornea curvature center A is indicated by a, a is obtained by extending the straight line CP toward the P side by the length r, and is thus expressed by the following equation.

$$a = \frac{p}{‖ p ‖} ( ‖ p ‖ + r ) \qquad ( 12 )$$

An optical axis vector is obtained by using the three-dimensional coordinate of the cornea curvature center A in the camera coordinate system and the three-dimensional coordinate of the eyeball center in the camera coordinate system.

Specifically, an optical axis vector g is a vector directed from the eyeball center E in the camera coordinate system toward the cornea curvature center A in the camera coordinate system, and is thus computed according to the following equation.

g=a−e (13)

As described above, the optical axis vector is defined, and a position and a posture of the eyeball model can be estimated through combination with an estimated value of the eyeball center coordinate.

As will be described below, the apparent pupil center computation portion 60 obtains a three-dimensional coordinate of the apparent pupil center in the camera coordinate system on the basis of the three-dimensional coordinate of the eyeball center in the camera coordinate system, the optical axis vector, and the predefined three-dimensional eyeball model.

First, a corner CEA formed between a line segment EA connecting the three-dimensional coordinate E of the eyeball center in the camera coordinate system to the three-dimensional coordinate A of the cornea curvature center in the camera coordinate system and a line segment EC connecting the three-dimensional coordinate E of the eyeball center in the camera coordinate system to the three-dimensional coordinate C of the image capturing unit 12 is calculated on the basis of the optical axis vector g and the three-dimensional coordinate e of the eyeball center in the camera coordinate system.

Specifically, in a case where an angle of the corner CED is indicated by ω, an angle of a corner DEA is indicated by ρ, and an angle of the corner CEA is indicated by \acute{ω}, the angle of the corner CEA is expressed by the following equation.

g=a−e (14)

Therefore, is obtained according to the following equation by using inner product computation.

$${ω}^{'} = arccos \frac{( - e ) \cdot g}{‖ - e ‖ ‖ g ‖} \qquad ( 15 )$$

A predefined angle correction amount corresponding to the calculated angle of the corner CEA is acquired.

Specifically, a relationship between an angle of the corner CEA and an angle difference p between the visual line vector g_{cr} obtained according to a corneal reflex method and the visual line vector g_{eb} obtained according to an eyeball model fitting method is obtained in advance, and the angle difference ρ corresponding to an angle of the corner CEA is obtained as a correction amount ρ by using the relationship.

A vector gₑb connecting the three-dimensional coordinate E of the eyeball center in the camera coordinate system to the three-dimensional coordinate A of the cornea curvature center in the camera coordinate system is computed on the basis of the three-dimensional coordinate e of the eyeball center in the camera coordinate system, the optical axis vector g, and the angle correction amount ρ according to the following equation.

$${g}_{eb} = g cos ρ + \frac{1}{sin ω} g cos ω sin ρ - \frac{‖ g ‖}{‖ e ‖ sin ω} e sin ρ \qquad ( 16 )$$

A point of which a distance is r from the cornea curvature center A is obtained on the vector g_{eb} connecting the three-dimensional coordinate E of the eyeball center in the camera coordinate system to the three-dimensional coordinate A of the cornea curvature center in the camera coordinate system.

Specifically, a distance between E and D is indicated by x. In a triangle EDA, since AE=u, AD=r, and an angle of the corner DEA=ρ, the following Equation (17) is established according to the cosine theorem. Equation (17) that is a quadratic equation of x is solved with respect to x, and a greater solution is used as the distance x between E and D.

r²=x²+u²−2xu cos ρ (17)

In a case where a three-dimensional vector of the pupil center D is indicated by d=dₓ, d_{y}, d_{z}), D is a point present at the distance x from E on the vector g_{eb}, and thus d is obtained according to the following Equation (18).

$$d = x \frac{{g}_{eb}}{‖ {g}_{eb} ‖} - e \qquad ( 18 )$$

The pupil center image coordinate computation portion 62 estimates an image coordinate of the pupil center in the face image on the basis of the three-dimensional coordinate of the apparent pupil center in the camera coordinate system.

Specifically, the three-dimensional coordinate of the apparent pupil center in the camera coordinate system is two-dimensionally projected by using camera parameters.

For example, in a case where a focal length represented in the pixel unit is indicated by f, and a coordinate of the image center is indicated by (x_{c},y_{c}), an image coordinate (Dₓ,D_{y}) of the pupil center is as follows.

$$\begin{matrix}{D}_{x} = f \frac{{d}_{x}}{{d}_{z}} + {x}_{c} & ( 19 ) \\ {D}_{y} = f \frac{{d}_{y}}{{d}_{z}} + {y}_{c} & ( 20 )\end{matrix}$$

An image coordinate of the pupil center can be estimated by using observed values of the corneal reflex through the process in each portion.

The structure parameter estimation unit 28 estimates a structure parameter of the three-dimensional eyeball model on the basis of the image coordinate of the corneal reflex estimated by the corneal reflex estimation unit 22 and the image coordinate of the pupil center estimated by the pupil estimation unit 24, to update the structure parameters of the three-dimensional eyeball model stored in the eyeball model storage portions 34 and 54 and also to obtain a visual line vector by using the estimated structure parameters of the three-dimensional eyeball model, and outputs the visual line vector from the output unit 16.

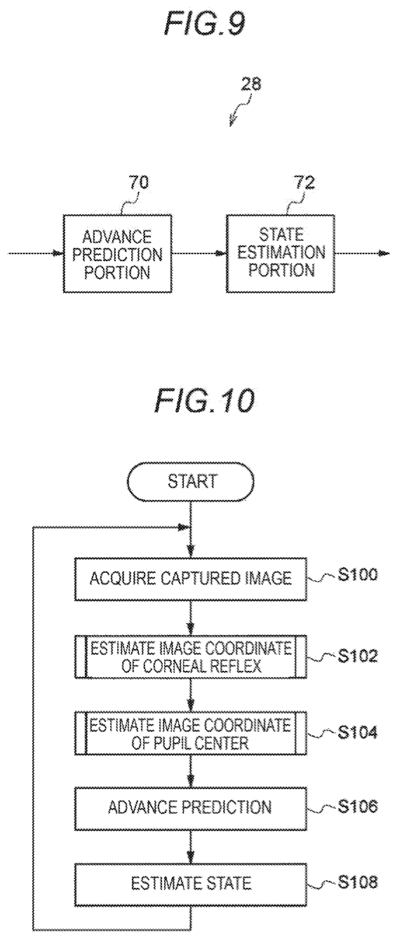

As illustrated in FIG. 9, the structure parameter estimation unit 28 is configured to include an advance prediction portion 70 and a state estimation portion 72.

Specifically, the advance prediction portion 70 and the state estimation portion 72 estimate structure parameters of the three-dimensional eyeball model by using a Kalman filter on the basis of the image coordinate observed value of the corneal reflex computed by the camera coordinate system corneal reflex computation portion 52, an image coordinate estimated value of the corneal reflex estimated by the corneal reflex estimation unit 22, an image coordinate observed value of the apparent pupil center computed by the apparent pupil center computation portion 32, an image coordinate estimated value of the apparent pupil center estimated by the pupil estimation unit 24, the optical axis vector g computed by the eyeball position/posture estimation portion 36, and the following Equation (21) for obtaining the optical axis vector g.

$$g = - a cos ( ϕ + θ ) + ( - \frac{cos ɛ}{\vert sin ɛ \vert} a + \frac{‖ a ‖}{‖ d ‖ \vert sin ɛ \vert} d ) sin ( ϕ + θ ) \\ ɛ = {cos}^{- 1} \frac{d \cdot a}{‖ d ‖ ‖ a ‖} \\ Here , \\ ϕ + θ = {sin}^{- 1} ( \frac{r}{s} \frac{{n}_{1}}{{n}_{2}} sin ( {cos}^{- 1} ( \frac{d \cdot p}{‖ d ‖ ‖ p ‖} ) + 2 {sin}^{- 1} ( \frac{‖ d - p ‖}{2 r} ) ) ) \\ - {sin}^{- 1} ( \frac{{n}_{1}}{{n}_{2}} sin ( {cos}^{- 1} ( \frac{d \cdot p}{‖ d ‖ ‖ p ‖} ) + 2 {sin}^{- 1} ( \frac{‖ d - p ‖}{2 r} ) ) ) + 2 {sin}^{- 1} ( \frac{‖ d - p ‖}{2 r} ) \qquad ( 21 )$$

Herein, a description will be made of an estimation principle using a Kalman filter.

In a case where the cornea curvature radius r, the distance u between the eyeball center and the cornea curvature center, and the distance s between the cornea curvature center and the pupil center that are time-invariant amounts are used as state variables, and D=(Dₓ,D_{y}), P=(Pₓ,P_{y}), g=(gₓ,g_{y},g_{z}) are used as observation variables, the following equation can be formed, and can be solved by using the Kalman filter.

Dₓ=f_{Dx}(r,u)

D_{y}=f_{Dy}(r,u)

Pₓ=f_{Px}(u)

P_{y}=f_{Px}(u)

gₓ=f_{gx}(r,s)

g_{y}=f_{gy}(r,s)

g_{z}=f_{gz}(r,s) (22)

Hereinafter, a state equation and an observation equation will be described in detail.

First, a description will be made of deriving of a state equation. A state equation of each of the cornea curvature radius r, the distance u between the eyeball center and the cornea curvature center, and the distance s between the cornea curvature center and the pupil center is expressed as follows.

rₜ=rₜ₋₁+∈ᵣ_{ₜ}

sₜ=sₜ₋₁+∈ₛ_{ₜ}

uₜ=uₜ₋₁+∈ᵤ_{ₜ}

Here, ∈ᵣ_{ₜ},∈ₛ_{ₜ}, ∈ᵤ_{ₜ} are system noise.

In a case where a state vector is indicated by xₜ=(rₜ,sₜ,uₜ)^{t}, the state equation is as follows.

xₜ=xₜ₋₁+∈ₜ

Next, a description will be made of deriving of an observation equation.

In a case where dₓ and d_{z} obtained according to Equation (18) are assigned to Equation (19), the following equation is obtained.

$${D}_{x} = f \frac{x \frac{{g}_{{eb}_{x}}}{‖ {g}_{eb} ‖} - {e}_{x}}{x \frac{{g}_{{eb}_{z}}}{‖ {g}_{eb} ‖} - {e}_{z}} + {x}_{c} = f \frac{{xg}_{{eb}_{x}} - {e}_{x} ‖ {g}_{eb} ‖}{{xg}_{{eb}_{z}} - {e}_{z} ‖ {g}_{eb} ‖} + {x}_{c} \qquad ( 23 )$$

Similarly, the following equation is obtained.

$${D}_{y} = f \frac{{xg}_{{eb}_{y}} - {e}_{y} ‖ {g}_{eb} ‖}{{xg}_{{eb}_{z}} - {e}_{z} ‖ {g}_{eb} ‖} + {x}_{c} \qquad ( 24 )$$

Here, x in Equations (23) and (24) is a greater solution when Equation (17) is solved with respect to x, and thus the following equation is obtained.

x=u cos ρ+{square root over ((u cos φ²−(u²−r²))} (25)

Here, eₓ, e_{y}, and e_{z} are obtained according to Equation (2). g_{eb}_{ₓ},g_{eb}_{_{y}},g_{eb}_{_{z}} are obtained by assigning Equations (13) and (12) to Equation (16).

As mentioned above, a formula can be established not to include Pₓ and P_{y} in the right sides of Equations (23) and (24), and only u and r are included as parameters in Equation (25) such that the following equations are obtained.

$$\begin{matrix}{D}_{x} = f \frac{{xg}_{{eb}_{x}} - {e}_{x} ‖ {g}_{eb} ‖}{{xg}_{{eb}_{z}} - {e}_{z} ‖ {g}_{eb} ‖} + {x}_{c} = {f}_{Dx} ( r , u ) & ( 26 ) \\ {D}_{y} = f \frac{{xg}_{{eb}_{y}} - {e}_{y} ‖ {g}_{eb} ‖}{{xg}_{{eb}_{z}} - {e}_{z} ‖ {g}_{eb} ‖} + {x}_{c} = {f}_{Dy} ( r , u ) & ( 27 )\end{matrix}$$

In a case where pₓ and p_{z} obtained according to Equation (8) are assigned to Equation (9), the following equation is obtained.

$${P}_{x} = f \frac{\frac{{a}_{x}}{‖ a ‖} ( ‖ a ‖ - r )}{\frac{{a}_{z}}{‖ a ‖} ( ‖ a ‖ - r )} + {x}_{c} = f \frac{{a}_{x}}{{a}_{z}} + {x}_{c} \qquad ( 28 )$$

In a case where aₓ and a_{z} obtained according to Equation (7) are assigned to Equation (27), the following equation is obtained.

$${P}_{x} = f \frac{{e}_{x} + u \frac{{g}_{x}}{‖ g ‖}}{{e}_{z} + u \frac{{g}_{z}}{‖ g ‖}} + {x}_{c} = f \frac{‖ g ‖ {e}_{x} + {ug}_{x}}{‖ g ‖ {e}_{z} + {ug}_{z}} + {x}_{c} \qquad ( 29 )$$

Similarly, the following equation is obtained.

$${P}_{y} = f \frac{‖ g ‖ {e}_{y} + {ug}_{y}}{‖ g ‖ {e}_{z} + {ug}_{z}} + {x}_{c} \qquad ( 30 )$$

Here, eₓ, e_{y}, and e_{z} are obtained according to Equation (2). g=(gₓ,g_{y},g_{z}) is obtained by assigning Equations (4) and (5) to Equation (6).

As mentioned above, a formula can be established not to include Dₓ and D_{y} in the right sides of Equations (29) and (30), and only u is included as a parameter such that the following equations are obtained.

$$\begin{matrix}{P}_{x} = f \frac{‖ g ‖ {e}_{x} + {ug}_{x}}{‖ g ‖ {e}_{z} + {ug}_{z}} + {x}_{c} = {f}_{Px} ( u ) & ( 31 ) \\ {P}_{y} = f \frac{‖ g ‖ {e}_{y} + {ug}_{y}}{‖ g ‖ {e}_{z} + {ug}_{z}} + {x}_{c} = {f}_{Py} ( u ) & ( 32 )\end{matrix}$$

Here, the optical axis vector g may be obtained according to two methods.

One is a first method in which the optical axis vector g is obtained by assigning Equations (4) and (5) to Equation (6). The other is a second method in which the optical axis vector g is obtained by using Equation (21).

A parameter is not included in the equation according to the first method, but the parameters r and s are included in the equation according to the second method.

Therefore, the optical axis vector g obtained by using the equation according to the first method is regarded as an observed value, and thus the following equation is obtained.

gₓ=f_{gx}(r,s)

g_{y}=f_{gy}(r,s)

g_{z}=f_{gz}(r,s)

In a case where an observation vector is indicated by y=(Dₓ,D_{y},Pₓ,P_{y},gₓ,g_{y},g_{z})^{t}, Equations (26), (27), (31), (32), (33), (34), and (35) are summarized as follows.

y=h(r,s,u)=(f_{Dx}(r,u),f_{Dy}(r,u),f_{Px}(u),f_{Py}(r,s),f_{gx}(r,s),f_{gy}(r,s),f_{gz}(r,s))^{t}

From the above description, the Kalman filter is as follows. First, a state equation is expressed as follows.

xₖ=xₖ₋₁+∈ₖ (36)

Here, ∈ₖ is a system noise vector.

An observation equation is expressed as follows.

yₖ=h(rₖ,sₖ,uₖ)+δₖ (37)

Here, δₖ is an observation noise vector.

In a prediction step, a state vector at the next time point is predicted according to the following equation.

\hat{x}_{k|k-1}=\hat{x}_{k-1|k-1}

\hat{P}_{k|k-1}=\hat{P}_{k-1|k-1} (38)

Here, Pₖ is a covariance matrix of an error.

In an update step, the state vector is updated according to the following equation.

$${e}_{k} = {z}_{k} - h ( {\hat{x}}_{k \vert k - 1} , 0 ) \\ {S}_{k} = {H}_{k} {P}_{k \vert k - 1} {H}_{k}^{T} + {R}_{k} \\ {K}_{k} = {P}_{k \vert k - 1} {H}_{k}^{T} {S}_{k}^{- 1} \\ {\hat{x}}_{k \vert k} = {\hat{x}}_{k \vert k - 1} + {K}_{k} {e}_{k} \\ {P}_{k \vert k} = ( I - {K}_{k} {H}_{k} ) {P}_{k \vert k - 1} \\ Here , {H}_{k} = \frac{\partial h}{\partial x} {\vert}_{{\hat{x}}_{k \vert k - 1}} \qquad ( 39 )$$

The advance prediction portion 70 predicts a state vector including structure parameters of the three-dimensional eyeball model by using equation (38) according to the above-described principle.

The state estimation portion 72 estimates a state vector according to Equation (39) on the basis of an observation vector including an image coordinate of the corneal reflex in the face image, estimated by the corneal reflex estimation unit 22 and an image coordinate of the pupil center in the face image, estimated by the pupil estimation unit 24, the state vector predicted by the advance prediction portion 70, and an observation equation representing an observation vector by using the state vector, and stores the cornea curvature radius r, the distance s between the cornea curvature center and the pupil center, and the distance u between the cornea curvature center and the eyeball center, included in the state vector, into the eyeball model storage portions 34 and 54.

The state estimation portion 72 obtains the optical axis vector g according to Equation (21), and outputs the optical axis vector g from the output unit 16.

Operation of Visual Line Measurement Apparatus

Next, a description will be made of an operation of the visual line measurement apparatus 10. First, when the irradiation unit 13 irradiates the eye of a subject with near infrared light, the image capturing unit 12 consecutively captures a face image of the subject.

A visual line measurement process routine illustrated in FIG. 10 is executed in the computer 14.

First, in step S100, the face image captured by the image capturing unit 12 is acquired.

In step S102, an image coordinate of a corneal reflex in the face image is estimated.

In step S104, an image coordinate of the pupil center in the face image is estimated.

In step S106, a state vector including structure parameters of a three-dimensional eyeball model at the present time is predicted according to Equation (38) by using an initial value of a structure parameter of the three-dimensional eyeball model or a state vector estimated in step S108 at the previous time.

In step S108, a state vector is estimated according to Equation (39) on the basis of an observation vector including the image coordinate of the corneal reflex estimated in step S102 and the image coordinate of the pupil center estimated in step S104, and an observation equation representing an observation vector by using a state vector, and the optical axis vector g is obtained according to Equation (21) by using structure parameters of the three-dimensional eyeball model included in the state vector and is then output from the output unit 16, and the flow returns to step S100.

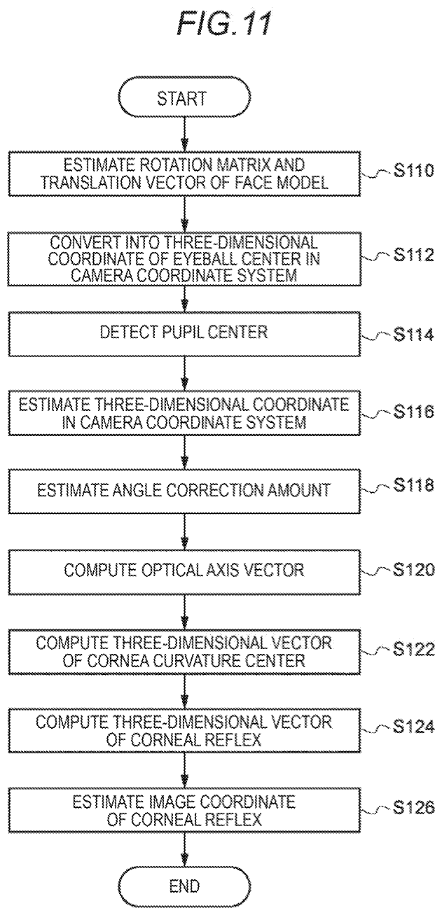

Step S102 is realized by a corneal reflex estimation process routine illustrated in FIG. 11.

First, in step S110, a face model is fitted to the current face image, and thus the current rotation matrix R and the current translation vector t of a face model coordinate system with respect to a camera coordinate system are obtained.

In step S112, a three-dimensional coordinate of the eyeball center in the face model coordinate system is converted into a three-dimensional coordinate of the eyeball center in the camera coordinate system by using the obtained rotation and translation vectors.

In step S114, the pupil center is detected from the face image, and a pupil center coordinate in an image coordinate system is obtained.

In step S116, a three-dimensional coordinate in the camera coordinate system is estimated on the basis of the image coordinate of the pupil center.

In step S118, since the image capturing unit 12 and the irradiation unit 13 are disposed such that an imaging direction of the image capturing unit 12 and an irradiation direction of the irradiation unit 13 are regarded to be present on the same axis, an angle correction amount is estimated by using an angle of a corner CED formed by a line segment connecting a three-dimensional coordinate of the apparent pupil center in the camera coordinate system to a three-dimensional coordinate of the eyeball center in the camera coordinate system and a line segment connecting the three-dimensional coordinate of the eyeball center in the camera coordinate system to a three-dimensional coordinate of the imaging unit.

In step S120, the three-dimensional optical axis vector g directed from a three-dimensional position of the eyeball center toward a three-dimensional position of the pupil center in the camera coordinate system is computed on the basis of the angle co of the corner CED, the three-dimensional coordinate e of the eyeball center in the camera coordinate system, and the angle correction amount.

In step S122, the three-dimensional coordinate a of the cornea curvature center A in the camera coordinate system is estimated by using the three-dimensional coordinate e of the eyeball center in the camera coordinate system, the optical axis vector g, and the distance u between the eyeball center E and the cornea curvature center A stored in the eyeball model storage portion 34.

In step S124, a point of which a distance is r from the cornea curvature center A on the vector CA is obtained, and a three-dimensional vector of the corneal reflex P is estimated.

In step S126, an image coordinate of the corneal reflex in the face image is estimated on the basis of the three-dimensional coordinate of the corneal reflex in the camera coordinate system.

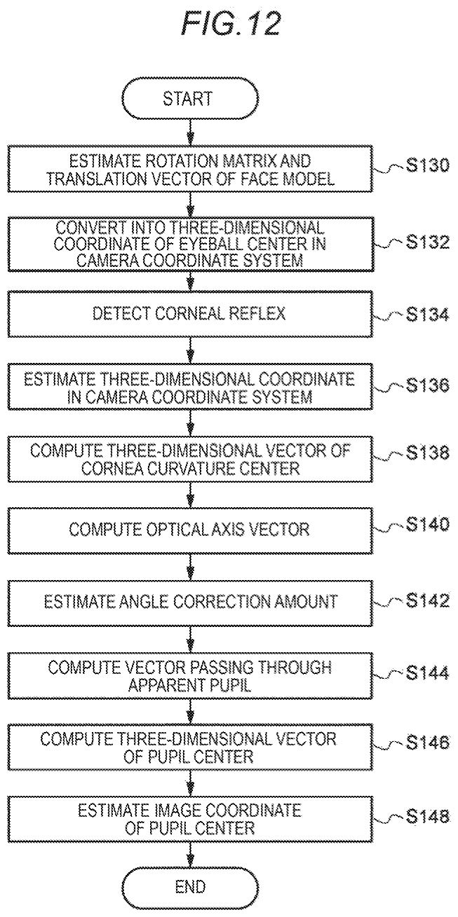

Step S104 is realized by a pupil center estimated process routine illustrated in FIG. 12.

First, in step S130, a face model is fitted to the current face image, and thus the current rotation matrix R and the current translation vector t of the face model coordinate system with respect to the camera coordinate system are obtained.

In step S132, a three-dimensional coordinate of the eyeball center in the face model coordinate system is converted into a three-dimensional coordinate of the eyeball center in the camera coordinate system by using the obtained rotation and translation vectors.

In step S134, the corneal reflex is detected from the face image, and a coordinate of the corneal reflex in the image coordinate system is obtained.

In step S136, a three-dimensional coordinate of the corneal reflex in the camera coordinate system is estimated by using the image coordinate of the corneal reflex.

In step S138, since the image capturing unit 12 and the irradiation unit 13 are disposed such that an imaging direction of the image capturing unit 12 and an irradiation direction of the irradiation unit 13 are regarded to be present on the same axis, a three-dimensional coordinate of the cornea curvature center A in the camera coordinate system is estimated by using the three-dimensional coordinate p of the corneal reflex in the camera coordinate system and the cornea curvature radius r.

In step S140, an optical axis vector is obtained by using the three-dimensional coordinate of the cornea curvature center A in the camera coordinate system and the three-dimensional coordinate of the eyeball center in the camera coordinate system.

In step S142, the corner CEA formed between the line segment EA connecting the three-dimensional coordinate E of the eyeball center in the camera coordinate system to the three-dimensional coordinate A of the cornea curvature center in the camera coordinate system and the line segment EC connecting the three-dimensional coordinate E of the eyeball center in the camera coordinate system to the three-dimensional coordinate C of the image capturing unit 12 is calculated on the basis of the optical axis vector g and the three-dimensional coordinate e of the eyeball center in the camera coordinate system, and a predefined angle correction amount corresponding to an angle of the calculated corner CEA is acquired.

In step S144, the vector g_{eb} connecting the three-dimensional coordinate E of the eyeball center in the camera coordinate system to the three-dimensional coordinate A of the cornea curvature center in the camera coordinate system is computed on the basis of the three-dimensional coordinate e of the eyeball center in the camera coordinate system, the optical axis vector g, and the angle correction amount ρ.

In step S146, a point of which a distance is r from the cornea curvature center A is obtained on the vector g_{eb} connecting the three-dimensional coordinate E of the eyeball center in the camera coordinate system to the three-dimensional coordinate A of the cornea curvature center in the camera coordinate system, and a coordinate of the point is used as a three-dimensional coordinate of the apparent pupil center in the camera coordinate system.

In step S148, an image coordinate of the pupil center in the face image is estimated on the basis of the three-dimensional coordinate of the apparent pupil center in the camera coordinate system.

As described above, the visual line measurement apparatus according to the embodiment disclosed here predicts a state vector including a structure parameter of an eyeball, and estimates a state vector on the basis of an observation vector including an estimated image coordinate of a corneal reflex in a face image and an estimated image coordinate of the pupil center in the face image, the predicted state vector, and an observation equation representing an observation vector by using the state vector, and can thus obtain the structure parameter of the eyeball with high accuracy in order to measure a visual line with a simple configuration.

By using the configuration of the visual line measurement apparatus according to the embodiment disclosed here, it is possible to obtain a correspondence relationship between an estimation formula (observation equation) for a corneal reflex and an apparent pupil center, and an observed value. Since a structure parameter is a fixed parameter that does not change over time in an observation equation, for example, a nonlinear Kalman filter is applied, and thus the structure parameter can be estimated such that an error between an observed value and an estimated value is the minimum.

In the corneal reflex method of the visual line measurement technique, a structure parameter including an individual difference of a three-dimensional eyeball model used therefor can be estimated without explicit calibration procedures, and thus the accuracy of estimating a visual line vector is improved without complex calibration procedures. Repetition calculation is not used for an estimation process, and thus the estimation process can be performed at a high speed. A fixed value is used as an estimated value, and thus it is hard to be influenced by wrong estimation due to an erroneous observed value.

An eyeball structure estimation apparatus according to an aspect of this disclosure includes an imaging unit that images a face of a person to be observed; a light irradiation unit that irradiates an eye of the person to be observed with light; a corneal reflex estimation unit that estimates an image coordinate of a corneal reflex in a face image representing the face imaged by the imaging unit by using structure parameters of an eyeball on the basis of a pupil center position of the eye in the face image; a pupil estimation unit that estimates an image coordinate of a pupil center in the face image by using the structure parameters of the eyeball on the basis of a position of the corneal reflex of the eye in the face image; an advance prediction unit that predicts a state vector including the structure parameters of the eyeball; and a state estimation unit that estimates the state vector on the basis of an observation vector including the image coordinate of the corneal reflex in the face image, estimated by the corneal reflex estimation unit, and the image coordinate of the pupil center in the face image, estimated by the pupil estimation unit, the state vector predicted by the advance prediction unit, and an observation equation representing the observation vector by using the state vector.

According to this disclosure, it is possible to obtain a structure parameter of an eyeball with high accuracy in order to measure a visual line with a simple configuration.

In the eyeball structure estimation apparatus according to the aspect of this disclosure, the observation vector may further include a visual line vector computed when the corneal reflex estimation unit estimates the image coordinate of the corneal reflex in the face image.

In the eyeball structure estimation apparatus according to the aspect of this disclosure, the structure parameters of the eyeball may include a cornea curvature radius of the eyeball, a distance between a cornea curvature center and the pupil center of the eyeball, and a distance between the cornea curvature center and an eyeball center.

In the eyeball structure estimation apparatus according to the aspect of this disclosure, a positional relationship between the imaging unit and the light irradiation unit, a positional relationship between the imaging unit and the eye, and a parameter regarding the imaging unit may satisfy a predefined constraint condition in which an imaging direction of the imaging unit and a light irradiation direction of the light irradiation unit are regarded to be present on the same axis.

In the eyeball structure estimation apparatus according to the aspect of this disclosure, the corneal reflex estimation unit may include a camera coordinate system eyeball center coordinate computation portion that estimates a three-dimensional coordinate of an eyeball center in a camera coordinate system on the basis of the face image representing the face imaged by the imaging unit, a pupil center computation portion that estimates a three-dimensional coordinate of an apparent pupil center in the camera coordinate system on the basis of the pupil center position of the eye in the face image, an eyeball position/posture estimation portion that computes a three-dimensional optical axis vector directed from a three-dimensional position of the eyeball center toward a three-dimensional position of the pupil center in the camera coordinate system on the basis of the three-dimensional coordinate of the eyeball center in the camera coordinate system and the three-dimensional coordinate of the apparent pupil center in the camera coordinate system, a corneal reflex computation portion that obtains a three-dimensional coordinate of the corneal reflex in the camera coordinate system on the basis of the three-dimensional coordinate of the eyeball center in the camera coordinate system, the optical axis vector, and a predefined three-dimensional eyeball model, and an image coordinate computation portion that estimates an image coordinate of the corneal reflex in the face image on the basis of the three-dimensional coordinate of the corneal reflex in the camera coordinate system.

In the eyeball structure estimation apparatus according to the aspect of this disclosure, the eyeball position/posture estimation portion may calculate a corner formed between a line segment connecting the three-dimensional coordinate of the apparent pupil center in the camera coordinate system to the three-dimensional coordinate of the eyeball center in the camera coordinate system and a line segment connecting the three-dimensional coordinate of the eyeball center in the camera coordinate system to a three-dimensional coordinate of the imaging unit, acquire a predefined angle correction amount corresponding to the formed corner, and compute a three-dimensional optical axis vector directed from the three-dimensional position of the eyeball center toward the three-dimensional position of the pupil center in the camera coordinate system on the basis of a vector connecting the three-dimensional coordinate of the eyeball center in the camera coordinate system to the three-dimensional coordinate of the apparent pupil center in the camera coordinate system, the acquired angle correction amount, the calculated formed corner, and the three-dimensional coordinate of the eyeball center in the camera coordinate system.

In the eyeball structure estimation apparatus according to the aspect of this disclosure, the pupil estimation unit may include a camera coordinate system eyeball center coordinate computation portion that estimates a three-dimensional coordinate of an eyeball center in a camera coordinate system on the basis of the face image representing the face imaged by the imaging unit, a corneal reflex computation portion that estimates a three-dimensional coordinate of the corneal reflex in the camera coordinate system on the basis of a position of the corneal reflex of the eye in the face image, an eyeball position/posture estimation portion that computes a three-dimensional optical axis vector directed from a three-dimensional position of the eyeball center toward a three-dimensional position of the cornea curvature center in the camera coordinate system on the basis of the three-dimensional coordinate of the eyeball center in the camera coordinate system and the three-dimensional coordinate of the corneal reflex in the camera coordinate system, a pupil center computation portion that obtains a three-dimensional coordinate of an apparent pupil center in the camera coordinate system on the basis of the three-dimensional coordinate of the eyeball center in the camera coordinate system, the optical axis vector, and a predefined three-dimensional eyeball model, and an image coordinate computation portion that estimates an image coordinate of the pupil center in the face image on the basis of the three-dimensional coordinate of the apparent pupil center in the camera coordinate system.

In the eyeball structure estimation apparatus according to the aspect of this disclosure, the pupil center computation portion may calculate a corner formed between a line segment connecting the three-dimensional coordinate of the eyeball center in the camera coordinate system to the three-dimensional coordinate of the cornea curvature center in the camera coordinate system and a line segment connecting the three-dimensional coordinate of the eyeball center in the camera coordinate system to a three-dimensional coordinate of the imaging unit on the basis of the optical axis vector and the three-dimensional coordinate of the eyeball center in the camera coordinate system, acquire a predefined angle correction amount corresponding to the calculated formed corner, and calculate a vector connecting the three-dimensional coordinate of the eyeball center in the camera coordinate system to the three-dimensional coordinate of the apparent pupil center in the camera coordinate system on the basis of the three-dimensional coordinate of the eyeball center in the camera coordinate system, the optical axis vector, and the angle correction amount, and obtain the three-dimensional coordinate of the apparent pupil center in the camera coordinate system on the basis of the calculated vector, the three-dimensional coordinate of the eyeball center in the camera coordinate system, and a predefined three-dimensional eyeball model.

As described above, the eyeball structure estimation apparatus of this disclosure can achieve an effect in which a structure parameter of an eyeball can be obtained with high accuracy in order to measure a visual line with a simple configuration.

The principles, preferred embodiment and mode of operation of the present invention have been described in the foregoing specification. However, the invention which is intended to be protected is not to be construed as limited to the particular embodiments disclosed. Further, the embodiments described herein are to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

What is claimed is:

1. An eyeball structure estimation apparatus comprising:
an imaging unit that images a face of a person to be observed;
a light irradiation unit that irradiates an eye of the person to be observed with light;
a corneal reflex estimation unit that estimates an image coordinate of a corneal reflex in a face image representing the face imaged by the imaging unit by using structure parameters of an eyeball on the basis of a pupil center position of the eye in the face image;
a pupil estimation unit that estimates an image coordinate of a pupil center in the face image by using the structure parameters of the eyeball on the basis of a position of the corneal reflex of the eye in the face image;
an advance prediction unit that predicts a state vector including the structure parameters of the eyeball; and
a state estimation unit that estimates the state vector on the basis of an observation vector including the image coordinate of the corneal reflex in the face image, estimated by the corneal reflex estimation unit, and the image coordinate of the pupil center in the face image, estimated by the pupil estimation unit, the state vector predicted by the advance prediction unit, and an observation equation representing the observation vector by using the state vector.

2. The eyeball structure estimation apparatus according to claim 1, wherein
the observation vector further includes a visual line vector computed when the corneal reflex estimation unit estimates the image coordinate of the corneal reflex in the face image.

3. The eyeball structure estimation apparatus according to claim 1, wherein
the structure parameters of the eyeball include a cornea curvature radius of the eyeball, a distance between a cornea curvature center and the pupil center of the eyeball, and a distance between the cornea curvature center and an eyeball center.

4. The eyeball structure estimation apparatus according to claim 1, wherein
a positional relationship between the imaging unit and the light irradiation unit, a positional relationship between the imaging unit and the eye, and a parameter regarding the imaging unit satisfy a predefined constraint condition in which an imaging direction of the imaging unit and a light irradiation direction of the light irradiation unit are regarded to be present on the same axis.

5. The eyeball structure estimation apparatus according to claim 1, wherein
the corneal reflex estimation unit includes
a camera coordinate system eyeball center coordinate computation portion that estimates a three-dimensional coordinate of an eyeball center in a camera coordinate system on the basis of the face image representing the face imaged by the imaging unit,
a pupil center computation portion that estimates a three-dimensional coordinate of an apparent pupil center in the camera coordinate system on the basis of the pupil center position of the eye in the face image,
an eyeball position/posture estimation portion that computes a three-dimensional optical axis vector directed from a three-dimensional position of the eyeball center toward a three-dimensional position of the pupil center in the camera coordinate system on the basis of the three-dimensional coordinate of the eyeball center in the camera coordinate system and the three-dimensional coordinate of the apparent pupil center in the camera coordinate system,
a corneal reflex computation portion that obtains a three-dimensional coordinate of the corneal reflex in the camera coordinate system on the basis of the three-dimensional coordinate of the eyeball center in the camera coordinate system, the optical axis vector, and a predefined three-dimensional eyeball model, and
an image coordinate computation portion that estimates an image coordinate of the corneal reflex in the face image on the basis of the three-dimensional coordinate of the corneal reflex in the camera coordinate system.

6. The eyeball structure estimation apparatus according to claim 5, wherein
the eyeball position/posture estimation portion
calculates a corner formed between a line segment connecting the three-dimensional coordinate of the apparent pupil center in the camera coordinate system to the three-dimensional coordinate of the eyeball center in the camera coordinate system and a line segment connecting the three-dimensional coordinate of the eyeball center in the camera coordinate system to a three-dimensional coordinate of the imaging unit,
acquires a predefined angle correction amount corresponding to the formed corner, and
computes a three-dimensional optical axis vector directed from the three-dimensional position of the eyeball center toward the three-dimensional position of the pupil center in the camera coordinate system on the basis of a vector connecting the three-dimensional coordinate of the eyeball center in the camera coordinate system to the three-dimensional coordinate of the apparent pupil center in the camera coordinate system, the acquired angle correction amount, the calculated formed corner, and the three-dimensional coordinate of the eyeball center in the camera coordinate system.

7. The eyeball structure estimation apparatus according to claim 1, wherein
the pupil estimation unit includes
a camera coordinate system eyeball center coordinate computation portion that estimates a three-dimensional coordinate of an eyeball center in a camera coordinate system on the basis of the face image representing the face imaged by the imaging unit,
a corneal reflex computation portion that estimates a three-dimensional coordinate of the corneal reflex in the camera coordinate system on the basis of a position of the corneal reflex of the eye in the face image,
an eyeball position/posture estimation portion that computes a three-dimensional optical axis vector directed from a three-dimensional position of the eyeball center toward a three-dimensional position of the cornea curvature center in the camera coordinate system on the basis of the three-dimensional coordinate of the eyeball center in the camera coordinate system and the three-dimensional coordinate of the corneal reflex in the camera coordinate system,
a pupil center computation portion that obtains a three-dimensional coordinate of an apparent pupil center in the camera coordinate system on the basis of the three-dimensional coordinate of the eyeball center in the camera coordinate system, the optical axis vector, and a predefined three-dimensional eyeball model, and
an image coordinate computation portion that estimates an image coordinate of the pupil center in the face image on the basis of the three-dimensional coordinate of the apparent pupil center in the camera coordinate system.

8. The eyeball structure estimation apparatus according to claim 7, wherein
the pupil center computation portion
calculates a corner formed between a line segment connecting the three-dimensional coordinate of the eyeball center in the camera coordinate system to the three-dimensional coordinate of the cornea curvature center in the camera coordinate system and a line segment connecting the three-dimensional coordinate of the eyeball center in the camera coordinate system to a three-dimensional coordinate of the imaging unit on the basis of the optical axis vector and the three-dimensional coordinate of the eyeball center in the camera coordinate system,
acquires a predefined angle correction amount corresponding to the calculated formed corner, and
calculates a vector connecting the three-dimensional coordinate of the eyeball center in the camera coordinate system to the three-dimensional coordinate of the apparent pupil center in the camera coordinate system on the basis of the three-dimensional coordinate of the eyeball center in the camera coordinate system, the optical axis vector, and the angle correction amount, and
obtains the three-dimensional coordinate of the apparent pupil center in the camera coordinate system on the basis of the calculated vector, the three-dimensional coordinate of the eyeball center in the camera coordinate system, and a predefined three-dimensional eyeball model.
